(19) United States
(12) Patent Application Publication
(10) Pub. No.: US 2026/0114856 A1
(43) Pub. Date: Apr. 30, 2026

(54) RELEASABLE COUPLING FOR A MEDICAL DEVICE

(71) Applicant: Boston Scientific Scimed, Inc., Maple Grove, MN (US)

(72) Inventors: Hao DANG, Brighton, MA (US); Juan Pablo ORTIZ GARCIA, Heredia (CR); Kevin WALSH, Marlborough, MA (US)

(73) Assignee: Boston Scientific Scimed, Inc., Maple Grove, MN (US)

(21) Appl. No.: 19/364,535

(22) Filed: Oct. 21, 2025

Related U.S. Application Data
(60) Provisional application No. 63/711,278, filed on Oct. 24, 2024.

(51) Int. Cl. A61B 10/06; A61B 10/02

(52) U.S. Cl. CPC A61B 10/06; A61B 2010/0208; A61B 2560/0425; A61B 2560/0443

(57) ABSTRACT

A medical device may comprise a handle having a handle body, a shaft, a wire, and a wire holder. The wire may extend through the shaft and the wire may be configured to actuate an end effector. The wire holder may be configured to removably couple the wire to the handle, and the wire holder may comprise a base having an opening and at least one actuation element having an extension. The at least one actuation element may be movable relative to the base, such that, in a first configuration, the extension may be outside of the opening and, in a second configuration, the extension may extend into the opening to define an aperture for removably coupling the wire to the wire holder.

### CROSS-REFERENCE TO RELATED APPLICATIONS

This application claims the benefit of priority to U.S. Provisional Application No. 63/711,278, filed on October 24, 2024, which is incorporated by reference herein in its entirety.

### TECHNICAL FIELD

The disclosure relates generally to couplings for medical devices. More specifically, aspects of the disclosure pertain to releasable couplings for attaching a proximal portion of a medical device to a distal portion of the medical device.

### BACKGROUND

Single-use medical devices can result in a large amount of waste, creating a burden on the environment. For example, many endoscopic devices, including, for example, hemostasis clips and biopsy forceps, are currently single-use devices. Such devices often include a handle, a shaft, and a distal tip that includes an end effector. The distal tip and the shaft are inserted in the body to perform a procedure at a tissue site (e.g., obtain a tissue sample), while the handle remains outside of the body. Disposal of the entire device creates substantial amounts of waste and environmental burden. Therefore, there is a need for an apparatus that results in less waste and environmental burden. The apparatus and methods described herein may alleviate this deficiency and one or more other deficiencies in the art. However, the attached claims specify the scope of this disclosure rather than the ability to solve any specific problem.

### SUMMARY

This disclosure includes devices and methods that allow for releasably coupling a proximal component of a medical device (including a handle) to a distal component of the medical device (including portions that are inserted in the body during use). Such devices and methods permit, for example, re-use of the proximal component with other distal components throughout the duration of a medical procedure, resulting in less waste. Each of the aspects disclosed herein may include one or more of the features described in connection with any of the other disclosed aspects. The examples described herein may have any of these features in any combination.

In an example, a medical device may comprise a handle having a handle body, a shaft, a wire, and a wire holder. The wire may extend through the shaft, and the wire may be configured to actuate an end effector. The wire holder may be configured to removably couple the wire to the handle, and the wire holder may comprise a base having an opening and at least one actuation element having an extension. The at least one actuation element may be movable relative to the base, such that, in a first configuration, the extension may be outside of the opening, and, in a second configuration, the extension may extend into the opening to define an aperture for removably coupling the wire to the wire holder.

Any examples described herein may have any of these features alone or in combination. The handle body may include a collet for removably coupling the shaft to the handle. The handle body may include an actuator for opening the collet to release the shaft from the handle. The actuator and collet may be biased to a closed position of the collet, in which the collet grips the shaft. The at least one actuation element may include two actuation elements. Each of the two actuation elements may have an extension, and each of the two actuation elements may be movable relative to the base, such that, in a first configuration, the extension of each of the two actuation elements may be outside of the opening and, in a second configuration, the extension of each of the two actuation elements may extend into the opening to define an aperture for removably coupling the wire to the wire holder. The two actuation elements may be on opposite sides of the base of the wire holder. The aperture may be smaller than the opening at the base of the wire holder. The wire may include a widened portion at a proximal end of the wire. The widened portion may be wider than the aperture, such that, in the second configuration, the widened portion may not pass through the aperture. The widened portion may be a first widened portion, and the wire may include a second widened portion, and, in the second configuration, the aperture may be positioned around the wire between the first widened portion and the second widened portion. The handle body may extend through the opening of the base. The at least one actuation element may include an opening for receiving a finger of a user. The opening may extend around the handle body, and a distal end of the wire may be coupled to the end effector. The wire holder may be movable proximally or distally relative to the handle body to move the wire proximally or distally.

In another example, a medical device may comprise a handle having a handle body, a shaft, a wire, and a wire holder. The wire may extend through the shaft and the wire may be configured to actuate an end effector. The wire holder may be configured to removably couple the wire to the handle, and the wire holder may comprise a base having an opening and a pair of actuation elements configured to transition the wire holder from a first configuration to a second configuration. In the first configuration, the wire may be removable from the wire holder. In the second configuration, the actuation elements may define an aperture that is smaller than the widened portion, such that the wire is fixed to the wire holder. According to some aspects, the handle body includes a collet for removably coupling the shaft to the handle.

In another example, a medical device may comprise a handle, a proximal wire extending through handle, a distal end of the proximal wire including a first connector, a shaft, a distal wire extending through the shaft, a proximal end of the distal wire including a second connector, a plug coupled to a proximal end of the shaft, and a socket coupled to a distal end of the handle. The socket may be configured to removably receive the plug. In a configuration in which the socket removably receives the plug, the proximal wire may be movable relative to the handle, and the distal wire may be movable relative to the shaft. According to some aspects, the socket may include a plurality of arms configured to releasably engage the plug to retain the plug within the socket. In addition, the plug and the socket may comprise a quick-connect system.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 illustrates a perspective view of an exemplary medical device, including an enlarged view of a distal portion of the medical device, according to an embodiment;

FIG. 2A illustrates a portion of a medical device, according to aspects of this disclosure;

FIG. 2B illustrates a perspective view of a wire holder of the medical device of FIG. 2A in an open configuration, according to aspects of this disclosure;

FIG. 2C illustrates a perspective view of the wire holder of FIG. 2B in a closed configuration, according to aspects of this disclosure;

FIG. 3A illustrates a perspective view of a portion of an exemplary medical device, according to aspects of this disclosure;

FIG. 3B illustrates an enlarged view of a portion of a coupler of the medical device of FIG. 3A, according to aspects of this disclosure;

FIG. 4 illustrates a perspective view of a portion of an exemplary medical device according to aspects of this disclosure; and

FIG. 5 illustrates a perspective view of a coupler for use with the medical devices described herein, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10% of a stated value.

Reference is now made in detail to examples of this disclosure, aspects of which are illustrated in the accompanying drawings. Wherever possible, the same reference numbers will be used throughout the drawings to refer to the same or like parts. Unless otherwise specified, aspects of the disclosed embodiments may be combined in any suitable manner.

Embodiments of this disclosure include a medical device that has a proximal component that releasably couples to a distal component. The proximal component may include a handle. The distal component may include at least a portion of a shaft, at least a portion of an actuation wire, and an end effector. The distal component may be insertable into a body lumen of a subject during a medical procedure. Prior to use of the medical device, at least a portion of the actuation wire may be releasably attached to an actuator of the handle, and a portion of the shaft may be releasably attached to a body of the handle. After use, the portion of the actuation wire may be de-coupled from the actuator, and the portion of the shaft may be de-coupled from body of the handle. In this way, the distal component, which contacts the patient during use, may be disposed of, and the proximal component may be cleaned as needed and re-used with a second distal component in a subsequent procedure.

Such embodiments of medical devices may result in reduced waste and reduced overall cost of the medical device (less handles needed, less packaging, etc.). Such embodiments also may result in a universal proximal component (including a handle) that may be used with numerous different types of distal components (including several types of end effectors).

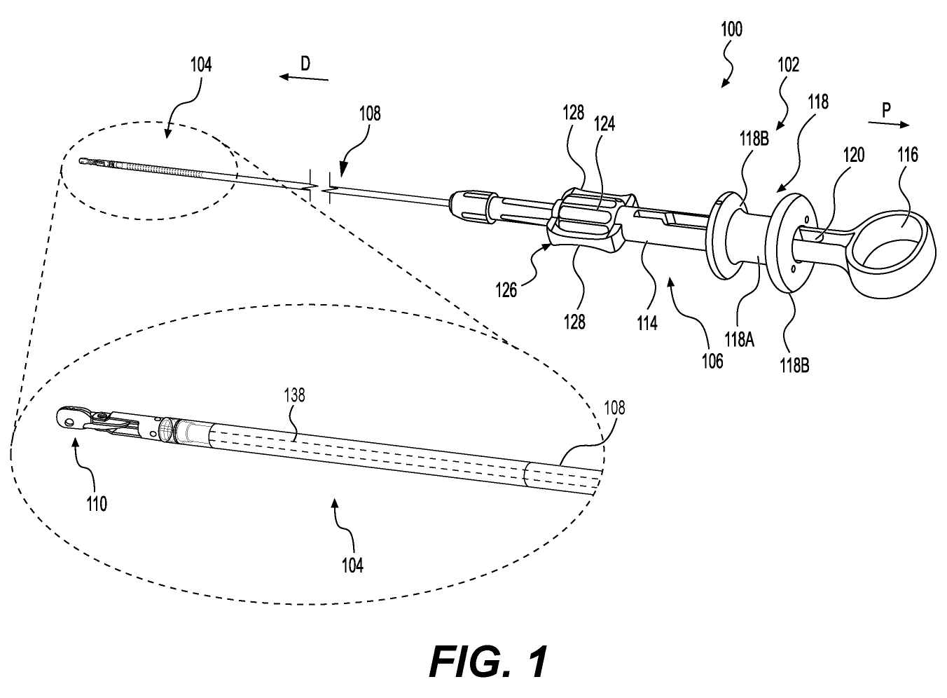

FIG. 1 illustrates a medical device 100, including a proximal portion 102 (i.e., a proximal component) and a distal portion 104 (i.e., a distal component). FIG. 1 includes an enlarged view of distal portion 104. As shown in FIG. 1, proximal portion 102 includes a handle 106. Distal portion 104 includes a shaft 108, for example, extending from a distal end of handle 106. Shaft 108 may include or otherwise radially surround a wire 138, for example, a pull wire or an actuation wire. For example, wire 138 may extend from handle 106 through shaft 108, and wire 138 may be movable (e.g., within shaft 108) via manipulation of one or more portions of handle 106 to control one or more aspects of distal portion 104 of medical device 100, for example, including an end effector 110. Although the term “wire” is used herein, it will be appreciated that wire 138 may include a cable or other similar type of member.

Medical device 100 includes end effector 110 at a distal end of shaft 108, for example, at distal portion 104. Medical device 100 may be delivered to the treatment site via an insertion device (e.g., through a working channel of an endoscope, ureteroscope, catheter, etc.). Although end effector 110 is shown as being a biopsy forceps, end effector 110 may be any type of endoscopic end effector (e.g., snare, basket, balloon, stent, clip, suturing device, stapler, etc.).

Handle 106 includes a main body 114, for example, including a ring 116 (e.g., a thumb ring), for example, at a proximal end of main body 114. Handle 106 also includes a first movable member or spool 118. Spool 118 may be an actuator and may include an indented portion 118A (e.g., with a relatively smaller lateral cross-section) and one or more (e.g., two) ridged or extended portions 118B (e.g., with a relatively larger lateral cross-section). In these aspects, indented portion 118A may receive one or more of the user’s fingers, such that movement of the user’s finger(s) controls the movement of spool 118. Main body 114 may include a slot 120, for example, extending longitudinally through a portion of main body 114, for example, from a position spaced distally from ring 116. A portion of spool 118 may extend into a portion of slot 120, such that spool 118 is movable (e.g., longitudinally movable distally and/or proximally) along slot 120. In these aspects, slot 120 may define a range of movement for spool 118. As discussed in detail below, spool 118 may be movable within slot 120, for example, proximally and/or distally, to control one or more aspects of end effector 110.

Additionally, handle 106 optionally includes a second movable member or knob 124. For example, main body 114 may include a cage 126, for example, formed by two arms 128 that partially surround knob 124. Knob 124 and cage 126 may be distal of slot 120 on main body 114. As discussed below, knob 124 may be an actuator and may be rotatable, for example, clockwise and/or counterclockwise, to control one or more aspects of end effector 110. For example, knob 124 may be rotatable about a longitudinal axis (e.g., a central longitudinal axis) of medical device 100.

Medical device 100 includes end effector 110 at distal portion 104. As shown, end effector 110 may be a forceps, for example, including two jaws. Nevertheless, this disclosure is not so limited, and end effector 110 may be any type of end effector, instrument, tool, or other device (e.g., grasper, snare, basket, hemostatic clip, stapler, ablation device, tome, suturing device, needle, knife, etc.).

End effector 110 is coupled to shaft 108 at the distal end of shaft 108. Wire 138 may be coupled to end effector 110, for example, via one or more end effector wires (not shown but having any of the properties of end effector actuators known in the art). In some aspects, the rotation of wire 138 about its axis, rotates end effector 110, and longitudinal movement of wire 138 controls or manipulates one or more aspects of end effector 110. For example, portions of end effector 110 may be fixedly coupled to a distal end of wire 138 (e.g., either directly or indirectly). As a result, movement of wire 138 (including distal wire portion 138B) may control the movement of the end effector 110.

For example, spool 118 may be moveably coupled to main body 114 of handle 106 via slot 120. In an embodiment, a portion of spool 118 may extend through (e.g., laterally) slot 120 and spool 118 may be movable along slot 120. The movement of spool 118 relative to main body 114 may actuate or control end effector 110. Spool 118 may translate within slot 120 in a first direction, towards a distal end of main body 114, and in a second direction, towards a proximal end of handle 106. The movement of spool 118 in these directions results in the actuation or de-actuation of end effector 110 (e.g., opening and closing).

The rotation of knob 124 may rotate wire 138, and consequently, end effector 110, around both the longitudinal axis of wire 138 and shaft 108. For example, when the user rotates knob 124 — relative to main body 114 and cage 126 — about its central longitudinal axis, wire 138 and end effector 110 also rotate. In use, rotating knob 124 clockwise results in a corresponding rotation of both wire 138 and end effector 110. Conversely, rotating knob 124 counterclockwise results in a corresponding counterclockwise rotation of both wire 138 and end effector 110. The rotation of knob 124, wire 138, and end effector 110, is independent of any rotation of shaft 108.

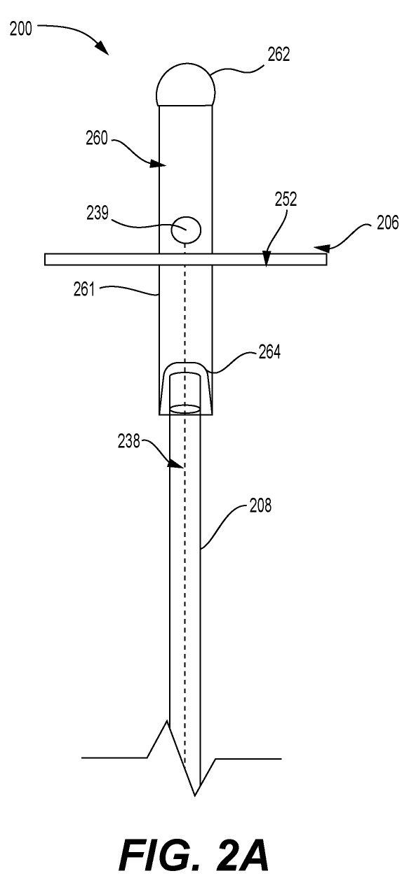

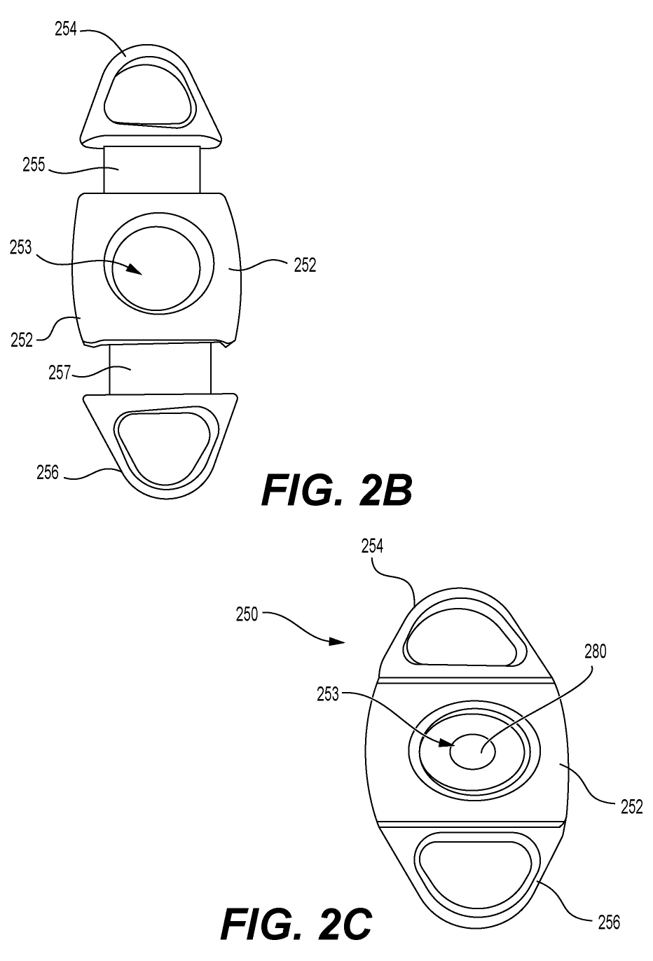

FIGS. 2A to 5 show mechanisms that are useful for releasably attaching distal components of a medical device (e.g., a shaft and an end effector) to a proximal component (e.g., handle having an actuator) of the medical device. For example, as described above, the embodiments may include a shaft and a wire that may be releasably or removably coupled to the handle of a medical device in different ways.

FIG. 2A depicts a portion of a medical device 200 similar to medical device 100, except as described herein. Medical device 200 may include a handle portion 206, similar to handle 106 and a shaft 208 (e.g., a sheath), similar to shaft 108. Shaft 208 may radially surround at least a portion of a wire 238, and wire 238 may extend through shaft 208. Additionally, wire 238 may extend proximally beyond the proximal-most end of shaft 208, where wire 238 may interface directly or indirectly with components, such as actuators, on handle portion 206. Similar to wire 138, wire 238 may actuate an end effector, having any of the properties of end effector 110.

As discussed, wire 238 may be similar to wire 138 or any other wire discussed below, except as described herein. FIG. 2A shows wire 238, which includes a widened portion 239. Widened portion 239 may have a larger (wider) cross-section than other portions of wire 238 and may be positioned at a proximal portion or proximal end of wire 238. In some examples, widened portion 239 may have an approximately spherical shape. The shape of widened portion 239 is not limited to a rounded configuration and may be any suitable shape, such as elliptical, prism-shaped, polygonal, or other shapes that may be received by a wire holder 250, described below. Widened portion 239 may be integral with wire 238 or separately formed and coupled to wire 238 using any suitable method known in the art, such as welding, adhesive bonding, or mechanical fastening. The dimensions of widened portion 239 may be tailored to provide a releasable connection with wire holder 250, as described below.

In some embodiments, wire 238 may include more than one widened portion 239. For example, one widened portion 239 may be positioned just proximal to wire holder 250 (as shown in FIG. 2A), while another widened portion 239 may be positioned just distal to wire holder 250. As wire 238 moves distally, wire holder 250 may contact a distal widened portion 239, and has wire holder 250 moves proximally, wire holder 250 may contact a proximal widened portion 239. Thus, widened portions 239 may retain wire holder 250 relative to wire 238.

Still referring to FIG. 2A, medical device 200 includes mechanisms used that facilitate the removable coupling of both shaft 208 and wire 238 to handle portion 206. Specifically, handle portion 206 includes a shaft holder 260 configured to removably couple shaft 208 to handle portion 206. Similarly, wire holder 250 may removably couple wire 238 to handle portion 206. Wire 238 may be movable relative to shaft holder 260 and to shaft 208, as discussed below.

Shaft holder 260 may be a coupling mechanism that includes a body 261, a collet 264, and an actuator 262 that helps removably couple shaft 208 to handle portion 206. Body 261 may house both collet 264 and actuator 262. Collet 264, or an alternative suitable gripping element, may be a cylindrical or semi-cylindrical component positioned within the distal end or distal portion of body 261. Collet 264 may be configured to expand outward, for example, to release shaft 108 or retract inward to grip shaft 208. The inner diameter of collet 264 may be a similar size as the outer diameter of shaft 208 to help form a secure fit when collet 264 is engaged. In some embodiments, the inner diameter of collet 264 may be adjustable, such that collet 264 may adjust to variations in the dimensions of shaft 208, helping collet 264 to accommodate shafts of different sizes.

Actuator 262 may be positioned at the proximal portion (e.g., proximal-most end) of shaft holder 260. In some embodiments, actuator 262 may be a button, lever, or sliding element that the user manipulates to control collet 264. Actuator 262 may also extend through body 261 and operate via a sliding or push-pull mechanism that engages and releases collet 264. For example, when a user pushes/pulls or otherwise manipulates actuator 262, actuator 262 may cause collet 264 to expand outward and open, forming a widened passage that facilitates the introduction or removal of shaft 208. For example, when collet 264 is open, a user may position shaft 208 within collet 264. Once shaft 208 is positioned within collet 264, a user may release or otherwise push/pull or manipulate actuator 262, which may cause collet 264 to retract inward and close, which may releasably couple shaft 208 to shaft holder 260.

In an embodiment, a biasing element (e.g., a spring or a coil) may be positioned within body 261, between collet 265 and the distal end of actuator 262. In these aspects, biasing element may impart a biasing force on actuator 262 toward an equilibrium position, which in turn imparts a biasing force on collet 265. For example, this biasing force may cause actuator 262 and collet 264 to retract inward in a default state, ensuring that shaft 208 remains securely coupled to handle portion 206 when not being manipulated by the user. In other words, actuator 262 and collet 264 may be biased to a closed position of collet 264, in which collet 264 grips shaft 208.

As depicted in FIG. 2B and FIG. 2C, wire holder 250 may be a coupling mechanism configured to removably couple wire 238 to handle portion 206. Wire holder 250 may include a base 252, an opening 253, a first actuation element 254, and a second actuation element 256 (i.e., a pair of actuation elements). Base 252 may be a central structure or platform for housing the other components of wire holder 250. Opening 253 may be centrally positioned through base 252. As described below, opening 253 may surround an aperture 280 that may be adjusted to be a smaller size than widened portion 239 of wire 238.

First actuation element 254 and second actuation element 256 may be on opposite sides of base 252. Each of actuation element 254 and actuation element 256 may include a finger opening (e.g., hole), which may be a suitable shape and size to enable a user to insert a finger through or otherwise position a finger on or within at least a portion of the slot. A user may manipulate actuation element 254 and actuation element 256 such that they expand radially outward from base 252 or move radially inward toward base 252. As shown in FIG. 2B, each of actuation element 254 and actuation element 256 may include an extension 255 and an extension 257, respectively. As shown in FIGS. 2B-2C, and described below, extension 255 and extension 257 may define the size of aperture 280

FIG. 2B shows an open configuration in which extension 255 and extension 257 are expanded outward from base 252. FIG. 2C shows a closed configuration in which at least a portion of extensions 255 and extension 257 are positioned within opening 253. Wire holder 250 may transition between an open configuration (FIG. 2B) and a closed configuration (FIG. 2C) using actuation element 254 and actuation element 256.

For example, to move from a closed configuration to an open configuration, the user may actuate actuation element 254 and actuation element 256 by applying an outward lateral force. As these elements move outwardly relative to base 252, extension 255 and extension 257 also transition outwardly, away from the area defined by opening 258. In other words, in a first configuration, extension 255 and extension 257 may be outside of opening 258. As extension 255 and extension 257 move outward from their positions in base 252 and opening 253, the diameter of aperture 280 may increase. Aperture 280 may provide adequate clearance for wire 238 to pass through to accommodate the insertion and removal of wire 238, including its widened portion 239.

Conversely, once wire 238 is inserted, wire holder 250 may be transitioned into the closed configuration shown in FIG. 2C. The user may move actuation element 254 and actuation element 256 inward toward base 252. Simultaneously, extension 255 and extension 257 are also driven inward toward and into opening 253 of base 252, defining aperture 280. Wire 238 may be received within aperture 280 in the closed configuration. Because widened portion 239 may have a larger diameter/width than aperture 280, aperture 280 may retain wire 238. In other words, in a second configuration, extension 255 and extension 257 may extend into opening 253 to define aperture 280 and for removably coupling wire 238 to wire holder 250. In some examples, a portion of wire 238 between two widened portions 239 may be received within aperture 280, allowing wire holder 250 to move proximally and distally while retaining wire 238.

In some aspects, as the user moves actuation element 254 and actuation element 256 inward toward base 252, the user may feel an increase in resistance, a subtle click, or another form of tactile or auditory feedback. The tactile feedback may signal that wire 238 is properly seated and removably coupled in place, helping the user to couple wire 238 to wire holder 250 without confirming the coupling visually. After completing a procedure, the user may transition to the open configuration (FIG. 2B) to release and replace wire 238.

Wire holder 250 may function as an actuator. In examples, as shown in FIG. 2A, body 261 may be received through opening 253 of wire holder 250. Body 261 may include slots that accommodate actuation elements 254 and 256. Wire 238 may extend through body 261 and into aperture 280 when wire holder 250 is in a closed configuration. A user may insert their fingers into the openings of actuation elements 254 and 256 and move the wire holder 250 proximally or distally along body 261. Thus, wire holder 250 may move wire 238 proximally or distally to actuate an end effector.

In some embodiments, alternative arrangements may exist between wire holder 250 and shaft holder 260. For example, wire holder 250 may be positioned alongside shaft holder 260, such that the proximal end (i.e., widened portion 239) of wire 238 extends outside of body 261 without being radially surrounded by shaft holder 260. In other words, wire holder 250 and shaft holder 260 may be side-by-side to facilitate independent coupling of both wire 238 and shaft 208 to their respective holders.

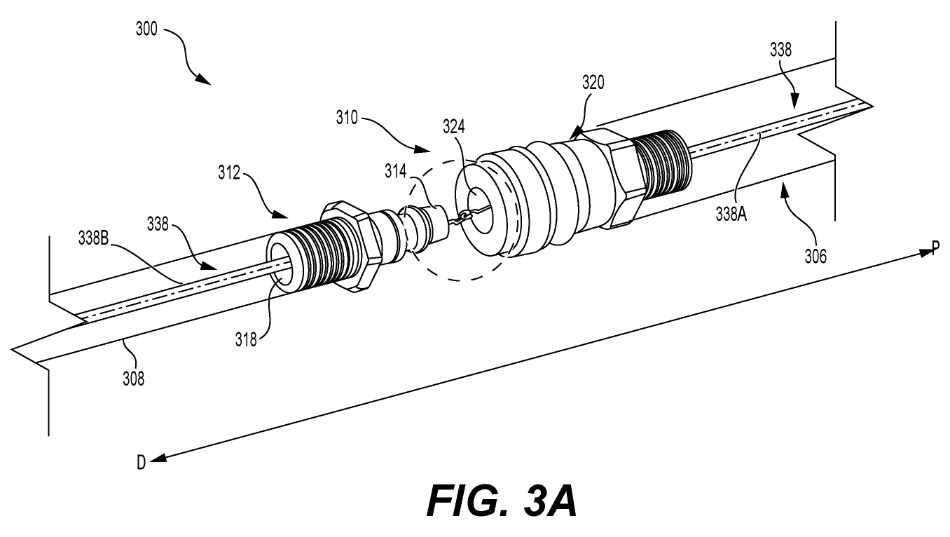

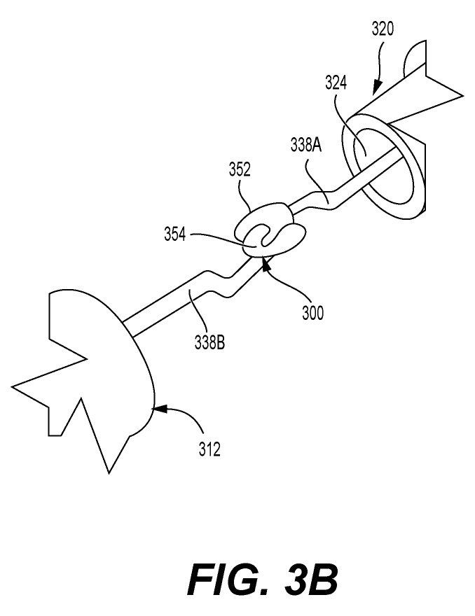

FIG. 3A shows a perspective view of a portion of an exemplary medical device 300 and a coupler 310. Medical device 300 may have any of the properties of medical device 100 unless otherwise specified herein. For example, medical device 300 may include a handle portion 306, which may be similar to handle 106. Handle portion 306 may include actuators like spool 118 and knob 124 that help transmit movement to the distal end of medical device 300 via wire 338.

Medical device 300 may include a shaft 308 (e.g., a sheath), similar to shaft 108, extending distally from handle portion 306. A wire 338 may include a proximal wire 338A and a distal wire 338B, which may be releasably coupled to one another as described below. In other words, wire 338 may be modular and composed of two portions. Shaft 308 may surround at least a portion of distal wire 338B. Distal wire 338B may extend proximally beyond the proximal end of shaft 308 to interface with components, such as actuators, located on handle portion 306 via proximal wire 338A. At its distal end, distal wire 338B may couple to an end effector similar to end effector 110. Distal wire 338B may extend the length of shaft 308 and may be movable within and relative to shaft 308 to transmit movement from the actuators on handle portion 306 to the end effector.

FIG. 3A also shows a coupler 310 that may removably couple the proximal components (e.g., handle portion 306) of medical device 300 to the distal components (e.g., shaft 308) of medical device 300, according to an exemplary embodiment. Coupler 310 may include a distal portion 312 fixedly coupled to shaft 308 and a proximal portion 320 fixedly coupled to handle portion 306.

Distal portion 312 of coupler 310 includes a plug 314 configured to removably couple to a complementary lumen 324 or socket in proximal portion 320. Alternatively, proximal portion 320 may include a plug, and distal portion 312 may include a lumen or socket. Distal portion 312 and proximal portion 320 may have properties of any quick-connect coupling known in the art. Plug 314 may be positioned at the proximal end of distal portion 312 and may have an elongated semi-cylindrical shape. Plug 314 may include a retention feature, such as a groove or ridge that engages with proximal portion 320 (e.g., a corresponding feature inside a lumen 324 of proximal portion 320.) Distal wire 338B may extend through a lumen 318 defined by distal portion 312, such that distal wire 338B is movable relative to lumen 318. Distal wire 338B may be movable within lumen 318 of distal portion 312 and relative to shaft 308 (i.e., which radially surrounds distal wire 338B). Distal wire 338B (e.g., similar to wire 138) may be coupled to an end effector, and the movement of distal wire 338B within lumen 318 may transmit mechanical forces to the end effector.

Proximal portion 320 includes lumen 324, which may function as a socket configured to retain and receive plug 314 of distal portion 312. For example, lumen 324 may have an internal shape aligning with (complementary to) a portion of plug 314. Insertion of plug 314 may produce a frictional or mechanical engagement with lumen 324 that helps portions of coupler 310 remain coupled to one another during use. The connection between distal portion 312 and proximal portion 320 may function as a quick-connect system, having any properties known in such systems, such as secure, reversible connections by way of a push-and-pull mechanism.

Proximal wire 338A may extend through lumen 324 of proximal portion 320 and interact with control mechanisms or functional components housed within handle portion 326, such as a spool and/or knob, as described above with respect to FIG. 1. Proximal wire 338A may move relative to handle portion 306 and lumen 324. As will be discussed below, in addition to plug 314 and lumen 324 functioning as a socket and plug system, proximal wire 338A and distal wire 338B also form aspects of coupler 310 and facilitate coupling proximal portion 320 and distal portion 312.

As shown in FIG. 3B, proximal wire 338A may include a hook 354 or a connector positioned at its distal end, while distal wire 338B may have a complementary hook 352 or connector positioned at its proximal end. Hook 354 of proximal wire 338A may extend distally past lumen 324 of proximal portion 320, while hook 352 of distal wire 338B may extend proximally of plug 314 of distal portion 312. Each hook 352, 354 may include a curved or semi-circular feature configured to engage with the corresponding hook on the opposing wire. Moreover, each hook 352, 354 may be immovable and remain fixed on the distal end of proximal wire 338A and distal wire 338B, respectively. As an alternative to hooks, other suitable coupling mechanisms such as magnetic attachments, snap-fit connections, or threaded engagements may be used.

As shown in FIG. 3B, hooks 352, 354 may engage with each other, thereby releasably coupling proximal wire 338A to distal wire 338B. In addition to assisting with coupling distal portion 312 and proximal portion 320, the connection between proximal wire 338A to distal wire 338B may facilitate the transmission of forces between proximal wire 338A and distal wire 338B.

As previously discussed, a distal component of device 300 (i.e., including shaft 308 and distal wire 338B) may be removably coupled to handle portion 306 via coupler 310. To assemble coupler 310, the user may align plug 314 of distal portion 312 with lumen 324 of proximal portion 320. The user may manually or automatically couple proximal wire 338A and distal wire 338B via hooks 352 and 354. Subsequently, distal portion 312, including plug 314, may be pushed into lumen 324 of proximal portion 320 until plug 314 is fully seated within lumen 324. In some aspects, a coupling between proximal wire 338A and distal wire 338B may be disposed within coupler 310.

Once the user has assembled coupler 310, the user may operate medical device 300 to perform a procedure. For example, the user may actuate aspects of handle portion 306 (e.g., a spool, knob, or other actuator) to transmit rotational or axial movement through proximal wire 338A and distal wire 338B, thereby actuating the end effector at the distal end of the device during the medical procedure. In the embodiment, distal wire 338B and proximal wire 338A may be movable relative to at least handle portion 306, shaft 408, and plug 414.

For disassembly, the process described above may be reversed. The user may pull or retract plug 314 away from lumen 324. As this occurs, hooks 350 and 352, may begin to disengage as plug 314 is further retracted or may be manually disengaged. The hooks may be moved in opposite directions until fully separated, thereby uncoupling proximal wire 338A from distal wire 338B. After disassembly, a distal component of medical device 300, including shaft 308, may be separated from handle portion 306 and a user may dispose of the distal component and reuse handle portion 306.

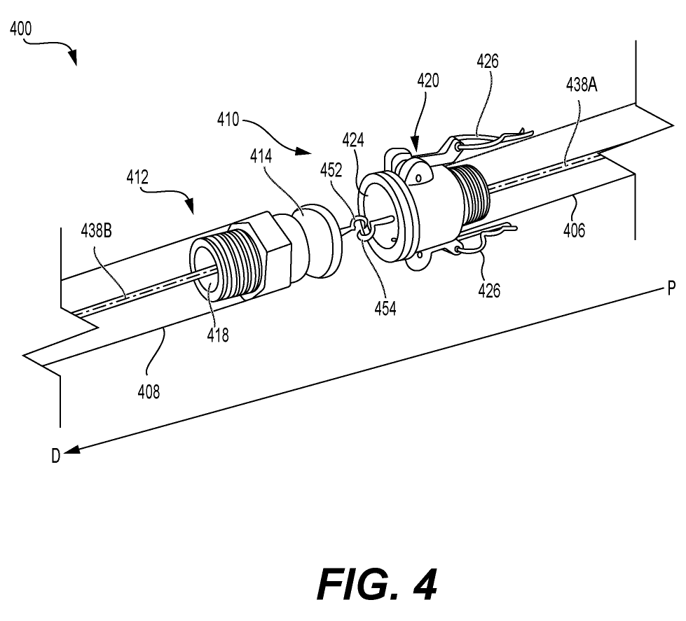

FIG. 4 depicts another embodiment for coupling the proximal and distal components of a medical device 400. Unless otherwise specified, the structure and methods of assembly and operation of this embodiment are similar to those described in FIGS. 3A-3B. For example, a coupler 410 includes a distal portion 412 and a proximal portion 420 that are similar to distal portion 312 and proximal portion 320, respectively.

However, in this embodiment, a coupler 410 may function as a cam and groove system. Accordingly, certain structural aspects of coupler 410 differ from the previous embodiment, incorporating features of cam and groove systems, such as those known in the art.

For example, medical device 400 may include a handle portion 406, which may be similar to handle portion 306 (e.g., similar to handle 106). Handle portion 406 may include actuators like spool 118 and knob 124 that help transmit movement to the distal end of medical device 400 via a wire 438. Similar to wire 338, wire 438 may include a proximal wire 438A and a distal wire 438B, which may be releasably coupled as described below. Moreover, medical device 400 may include a shaft 408, similar to shaft 308, detachably extending distally from handle portion 406 and radially surrounding at least a portion of distal wire 438B.

Distal wire 438B extends the length of shaft 408 and may move within shaft 408. At the distal end, distal wire 438B may be coupled to an end effector similar to end effector 110. Similar to the wires disclosed above, wire 438 may also transmit longitudinal or rotational movement from the actuators on handle portion 406 (e.g., spools, knobs, or other actuators) to the end effector.

FIG. 4 also depicts a coupler 410 similar to coupler 310 that may releasably attach or couple the proximal components (e.g., handle portion 406) of medical device 400 to the distal components (e.g., shaft 408) of medical device 400. Distal portion 412 of coupler 410 may be fixedly coupled to shaft 408 and proximal portion 420 of coupler 410 may be fixedly coupled to handle portion 406.

Distal portion 412 of coupler 410 includes a plug 414 (e.g., similar to plug 314) configured to removably couple with a complementary socket or lumen 424 in proximal portion 420. Distal wire 438B may extend through a lumen 418 defined by distal portion 412. Distal wire 438B may be movable within lumen 418 of distal portion 412 and relative to shaft 408 (i.e., which radially surrounds wire 438B).

Proximal portion 420 includes lumen 424, similar to lumen 324, which may also function as a socket configured to receive plug 414 of distal portion 412. The connection or coupling between plug 414 and lumen 424 may be similar to the connection between plug 314 and lumen 324 described in FIGS. 3A-3B. However, in this embodiment, distal portion 412 and proximal portion 420 also operate as part of a cam and groove connection system, incorporating properties known in such systems to achieve secure and reliable engagement.

For example, proximal portion 420 may include a plurality of arms 426, such as two arms positioned on opposite sides of proximal portion 420. Arms 426 may be pivotable elements that may be rotated (e.g., rotated around a pivot point/axis) to engage with complementary features on distal portion 412. Particularly, arms 426 may engage an outer surface of distal portion 412 or a groove or gasket on the outer surface of distal portion 412. Arms 426 may produce a clamping action that help plug 414 remain positioned within lumen 424 during operation. Additionally, arms 426 may compress the gasket on distal portion 412 and form a seal between distal portion 412 and proximal portion 420. In other words, arms 426 may be configured to releasably engage plug 414 to retain plug 414 within socket or lumen 424.

Proximal wire 438A may extend through lumen 424 of proximal portion 420 and interact with control mechanisms or functional components housed within handle portion 406 (e.g., actuators, such as knobs or spools). Proximal wire 438A may move relative to handle portion 406 and lumen 424. As will be discussed below, proximal wire 438A and distal wire 438B also form aspects of coupler 410 and facilitate coupling proximal portion 420 and distal portion 412.

Proximal wire 438A may include a hook 454 positioned at its distal end, while distal wire 438B may have a complementary hook 452 positioned at its proximal end. As shown in FIG. 4 and described above with respect to FIGS. 3A-3B, hooks 452, 454 may engage with one another, thereby releasably coupling proximal wire 438A to distal wire 438B. In addition to assisting with coupling distal portion 412 and proximal portion 420, the coupling between proximal wire 438A to distal wire 438B may facilitate the transmission of forces between distal wire 438B and proximal wire 438A.

As previously discussed, distal portion 412 (including shaft 408 and distal wire 438B) is releasably coupled to handle portion 406 via coupler 410. The assembly may be similar to the embodiment of FIGS. 3A-3B. To assemble coupler 410, the user may align plug 414 of distal portion 412 with lumen 424 of proximal portion 420. Once aligned, proximal wire 438A and distal wire 438B may be interlocked as hook 452 engages with hook 454. After hook 452 and hook 454 are engaged, distal portion 412, including plug 414, may be pushed into lumen 418 of proximal portion 420 until plug 414 is fully seated within lumen 424.

As discussed, proximal portion 420 may include arms 426. Once plug 414 is seated within lumen 424, the user may rotate ends of arms 426 toward distal portion 412. As the user rotates arms 426, the ends of arms 426 may engage grooves or press against the gasket of distal portion 42, creating a clamping action that helps secure plug 414 into lumen 424 and may also form a seal that helps prevent fluid leakage. Once the user has assembled coupler 410, the user may operate medical device 400 to perform a procedure. For example, actuating aspects of handle portion 406 to transmit rotational or axial movement through proximal wire 438A and distal wire 438B, thereby actuating the end effector at the distal end of the device during the medical procedure.

The disassembly may be similar to the embodiment of FIGS. 3A-3B. The user may disengage arms 426, for example, rotating the arms in the opposite directions from the coupling position (moving free ends of arms 426 proximally), which releases the clamping force applied to distal portion 412 and plug 414 and disengages arms 426 from distal portion 412. Subsequently, the process may be the same as the process described above for the embodiment of FIGS. 3A-3B. For example, pulling or retracting plug 414 away from lumen 424 and disengaging hooks 452 and 454 until distal portion 412 and proximal portion 420 are disassembled.

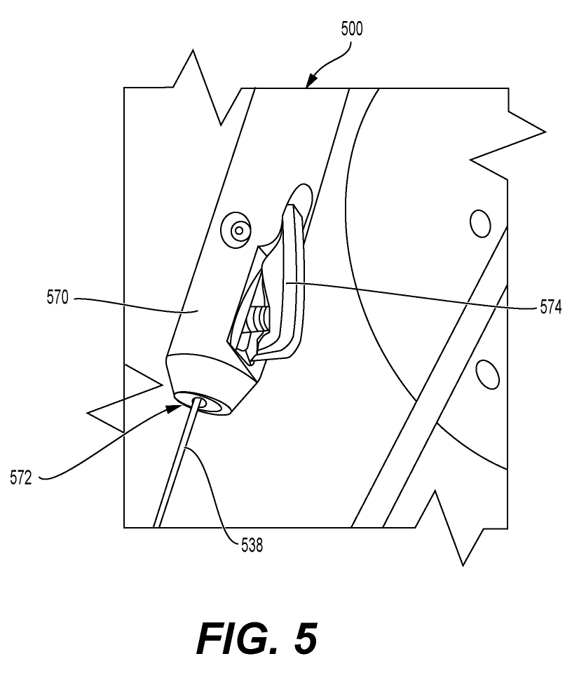

FIG. 5 illustrates an embodiment of a wire coupler 500, which facilitates the coupling of a wire 538 to a control mechanism, such as an actuator. Wire coupler 500 may be used in place of or alongside the couplers disclosed above. Wire coupler 500 includes a body 570, a lumen 572, and an arm 574. Body 570 is configured to receive and hold a wire 538 within lumen 572. Wire coupler 500 also includes arm 574 coupled to body 570 and may function as a coupling mechanism.

Arm 574 engages with wire 538, securing it within lumen 572 by applying a clamping force or other mechanical engagement. In this embodiment, arm 574 may resemble a clip or latch mechanism, similar to a bullet clip having any properties of bullet clips known in the art. Once wire 538 is positioned in lumen 572, arm 574 may be actuated or pushed into place, helping locking wire 538 remain coupled within body 570. Once wire 538 is secured, wire 538 may be actuated in a similar manner as any of the wires described above (e.g., wire 138). To release wire 538, the user may reverse the motion of arm 574, disengaging the locking mechanism so wire 538 may be removed from body 570.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle having a handle body;
a shaft;
a wire extending through the shaft, wherein the wire is configured to actuate an end effector; and
a wire holder configured to removably couple the wire to the handle, the wire holder comprising:
a base having an opening; and
at least one actuation element having an extension, wherein the at least one actuation element is movable relative to the base, such that, in a first configuration, the extension is outside of the opening and, in a second configuration, the extension extends into the opening to define an aperture for removably coupling the wire to the wire holder.

2. The medical device of claim 1, wherein the handle body includes a collet for removably coupling the shaft to the handle.

3. The medical device of claim 2, wherein the handle body includes an actuator for opening the collet to release the shaft from the handle.

4. The medical device of claim 3, wherein the actuator and collet are biased to a closed position of the collet, in which the collet grips the shaft.

5. The medical device of claim 1, wherein the at least one actuation element includes two actuation elements, wherein each of the two actuation elements has an extension, wherein each of the two actuation elements is movable relative to the base, such that, in a first configuration, the extension of each of the two actuation elements is outside of the opening and, in a second configuration, the extension of each of the two actuation elements extends into the opening to define an aperture for removably coupling the wire to the wire holder.

6. The medical device of claim 5, wherein the two actuation elements are on opposite sides of the base of the wire holder.

7. The medical device of claim 5, wherein the aperture is smaller than the opening in the base of the wire holder.

8. The medical device of claim 1, wherein the wire includes a widened portion at a proximal end of the wire.

9. The medical device of claim 8, wherein the widened portion is wider than the aperture, such that, in the second configuration, the widened portion cannot pass through the aperture.

10. The medical device of claim 8, wherein the widened portion is a first widened portion, wherein the wire includes a second widened portion, and wherein, in the second configuration, the aperture is positioned around the wire between the first widened portion and the second widened portion.

11. The medical device of claim 1, wherein the opening extends around the handle body.

12. The medical device of claim 1, wherein a distal end of the wire is coupled to the end effector.

13. The medical device of claim 1, wherein the wire holder is movable proximally or distally relative to the handle body to move the wire proximally or distally.

14. The medical device of claim 10, wherein the at least one actuation element includes an opening for receiving a finger of a user.

15. The medical device of claim 10, wherein the handle body extends through the opening of the base.

16. A medical device, comprising:
a handle having a handle body;
a shaft;
a wire extending through the shaft, wherein the wire is configured to actuate an end effector, and wherein a proximal end of the wire includes a widened portion; and
a wire holder configured to removably couple the wire to the handle, the wire holder comprising:
a base having an opening; and
a pair of actuation elements configured to transition the wire holder from a first configuration to a second configuration, wherein, in the first configuration, the wire is removable from the wire holder, and wherein, in the second configuration, the actuation elements define an aperture that is smaller than the widened portion, such that the wire is fixed to the wire holder.

17. The medical device of claim 16, wherein the handle body includes a collet for removably coupling the shaft to the handle.

18. A medical device, comprising:
a handle;
a proximal wire extending through the handle, a distal end of the proximal wire including a first connector;
a shaft;
a distal wire extending through the shaft, a proximal end of the distal wire including a second connector;
a plug coupled to a proximal end of the shaft; and
a socket coupled to a distal end of the handle, wherein the socket is configured to removably receive the plug, wherein in a configuration in which the socket removably receives the plug, the proximal wire is movable relative to the handle, and the distal wire is movable relative to the shaft.

19. The medical device of claim 18, wherein the socket includes a plurality of arms configured to releasably engage the plug to retain the plug within the socket.

20. The medical device of claim 18, wherein the plug and the socket comprise a quick-connect system.
